(19) United States
(12) Patent Application Publication
(10) Pub. No.: US 2012/0135924 A1
(43) Pub. Date: May 31, 2012

(54) Prosthesis for Joint Cartilage Repair and Method of Manufacture

(75) Inventors: Guo-Feng Xu, Guangzhou (CN); Bin Xu, Guangzhou (CN)

(73) Assignee: GRANDHOPE BIOTECH CO., LTD., Guangzhou (CN)

(21) Appl. No.: 13/363,089

(22) Filed: Jan. 31, 2012

Related U.S. Application Data
(63) Continuation of application No. 12/319,622, filed on Jan. 8, 2009.
Application No. 12/319,622 is a continuation-in-part of application No. 11/494,817, filed on Jul. 28, 2006.

(30) Foreign Application Priority Data
Jul. 22, 2008 (CN) 200810029653

(51) Int. Cl. A61K 47/46; A61P 19/04; C12S 3/00; A61K 38/03

(52) U.S. Cl. 514/7.6; 514/773; 435/267

(57) ABSTRACT

A cartilage prosthesis is made according to a method that includes the steps of collecting animal material from a bovine, ovine or porcine source, the animal material being a cartilage, shaping the animal material to provide a desired shape for the cartilage implant, removing cells from the animal material, crosslinking the animal material, removing antigens from the animal material, subjecting the animal material to an alkaline treatment, coupling into the animal material active substances which are capable of adhering growth factor and stem cell, and packing the animal material in a container that contains a sterilization solution.

### BACKGROUND OF THE INVENTION

1. Field of the Invention

The present invention relates to a medical prosthesis for human implantation, and in particular, to biological prosthesis that is used for the repair and treatment of joint cartilage damage.

2. Description of the Prior Art

Joint cartilage pathology and damage are frequently seen in orthopedic diseases, and may cause joint movement and function to be impeded and make walking extremely painful, thereby having a serious impact on a patient's work and daily life. To date, there is no ideal treatment method.

Under one type of treatment, a small number of orthopedic surgeons perform autologous transplantation treatment with a bone-cartilage shaft (for example, the autologous hip bone lateral bone-cartilage shaft). Unfortunately, secondary pathology frequently arises at the bone donation site to the extent that it is lost, which causes the patient to regress.

Another type of treatment involves the use of homologous (cadaver) bone cartilage transplants for treatment, but this treatment is unreliable because of unsolved immune rejection problems. In addition, because of ethical issues, viral transfection and other problems, it is rarely used.

Heterologous bone-cartilage transplant is even more problematic because the processing technology is not yet acceptable, and problems relating to immune rejection, and eradication of viruses from animal source materials, have yet to be solved, so this type of treatment is not desirable.

In light of the above, joint cartilage damage repair is still in a stagnant state today in which unsuitable materials are being used. Thus, there still remains a need for a biological prosthesis that is suitable for use in joint cartilage repair, and which avoids the drawbacks described above.

### SUMMARY OF THE DISCLOSURE

In order to accomplish the objects of the present invention, the present invention provides a joint cartilage repair piece made according to a method that comprises the following steps:

collecting animal material from a bovine, ovine or porcine source, the animal material being a cartilage;

shaping the animal material to provide a desired shape for the cartilage implant;

removing cells from the animal material;

crosslinking the animal material;

removing antigens from the animal material; and

coupling into the animal material active substances which are capable of adhering growth factor and stem cell.

The biological joint cartilage repair piece of the present invention provides a suitable material for joint cartilage damage repair. This joint cartilage repair material is sourced using wide-ranging heterologous bone-cartilage shafts as its raw material, which is then processed for the manufacture of bone and cartilage substrates (interstitial). After the bone-cartilage shaft undergoes the processing steps of the present invention, it has the advantages of effective immunogen removal, biocompatibility, and the ability to adhere and release and transmit a plethora of body self-repair growth factors and stem cells to the damaged site after implantation. The growth factors and stem cells are used to promote bone-cartilage regenerative repair; for example, fibroblast growth factor (FGF), transforming growth factor β (TGF β), insulin-like growth factor (IGF), platelet-derived growth factor (PDGF), bone morphogenetic proteins (BMP), bone marrow stem cells, etc., thereby achieving highly effective expression at the implant over a long period of time, inducing bone-cartilage tissue regeneration, and ultimately resulting in regenerative repair.

### BRIEF DESCRIPTION OF THE DRAWINGS

FIG. 1 is a perspective view of a cylindrical-shaped cartilage repair piece according to the present invention, with the cartilage layer indicated by “A” and the bone pedestal under the cartilage layer indicated by “B”.

FIG. 2 is a perspective view of a rectangular-shaped cartilage repair piece according to the present invention.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The present invention provides a method for producing a biological joint cartilage repair piece used for regenerative repair of joint cartilage damage. The specific technical workflow process for preparation is as follows:

1. Pretreatment (sterilization and removal of foreign matter)

2. Cut to obtain cartilage with a bone pedestal

3. Cell removal

4. Crosslinking and fixation

4a. NaOH Treatment (only if from a bovine or ovine source)

5. Multiform antigen removal

6. Technical treatment of tissue induction

7. Washing and cleaning

8. Sealing and packaging

9. Sterilization and virus eradication

Step 1: In the pretreatment step, the material is directly obtained by cutting the cartilage with a bone pedestal from porcine, bovine or ovine joints using techniques that are well-known in the art. The material is immersed and sterilized in broad-spectrum antibacterial agents, and impurities such as tendon and muscle tissue are removed by stripping them off using techniques that are well-known in the art.

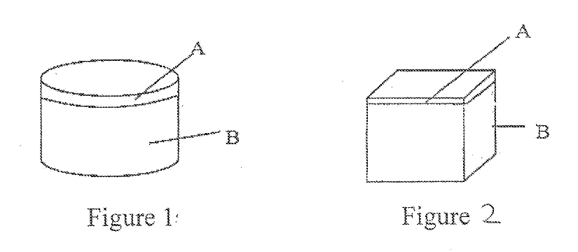

Step 2: The material is cut using well-known tools and methods into the desired shapes, such as those shown in FIGS. 1 and 2, where A is the cartilage layer and B is the bone pedestal.

Step 3: The cell removal step involves the use of an enzymatic method or a detergent elution method to remove the cells; either or both methods may be used. For the enzymatic method, pepsin or trypsin, or a mixture of the two, can be used to perform enzymatic destruction of cells. In the detergent elution method, after freezing loosened tissue containing cells, hypertonic or hypotonic detergents (also called surfactant) are used to detach the cells. The detergents that can be used are Triton X100, Tween-20 and OP-10.

Step 4: The crosslinking and fixation step involves use of epoxide compound crosslinking to fix the protein molecules in the bone and cartilage substrate (also called interstitial), making them stable, so they are not susceptible to deterioration or degradation by microorganisms. The epoxy used is expressed by the following formula

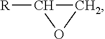

here R=CₙH₂ₙ₊₁-group or

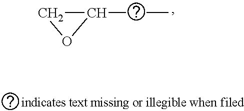

here n =0, 1, 2 . . . 12. The reagent concentration is between 0.1-2N, and the reaction temperature is selected between 5-40° C. The reaction time is set as needed for stability (the longer the time the higher the stability, so it is not susceptible to degradation), and is generally between 8 to 96 hours.

Step 5: According to modern immunological theory, the antigenicity of animal tissues stems mainly from active groups located at specific sites and in specific conformations, and these active groups include —H₂*,—OH*, —SH*, etc. The specific conformations result mainly from some specific hydrogen bonding formed by spiral protein chains. The specific sites and conformations are called antigen determinants. The antigen removal step uses multiple reagents to block the active groups and alter the specific conformation. The reagents used to block specific active groups are mainly nucleophilic reagents that react easily with —NH₂*,—OH*, —SH* and other similar groups. These reagents include carboxylic acid anhydrides, acyl chlorides, acylamides, epoxy compounds, etc. The reagents that can be used to alter specific conformations include class one strong hydrogen bond formation agents, such as guanidine hydrochloride. Because the specific conformations result mainly from some specific hydrogen bonding formed by spiral protein chains, using strong hydrogen bond formation agents to replace the specific hydrogen bond makes it possible to change the specific conformation. Here the * symbol on the groups indicates that they are a small number of specific groups which are located in specific locations and are able to produce a response to immune signals, and they are not the standard —NH₂, —OH, —SH groups. These specific groups are in a high-energy activity state, preferable for nucleophilic reagent initiated reactions, just as the catalyst's active center is preferable for the reactant or toxin reaction.

Step 6: The technical treatment of tissue induction involves coupling an active substance capable of adhering growth factors or stem cells to facilitate the accumulation of growth factors and stem cells released by the self-repair mechanism of the body on the implant and delivering them to the wound area, while facilitating high expression for a long period of time and promoting bone pedestal and cartilage repair. The active substances introduced can include some specific polypeptides or glycosaminoglycan compounds. The main specific polypeptides are mainly class-one containing 16 lysines with arginine, glycine, aspartic acid, and other polypeptides, for example, a lysine (16)—glycine-arginine-glycine-aspartic acid-serine-proline-cysteine polypeptide. The glycosaminoglycan compounds can include hyaluronic acid, chondroitin sulfate, cortisone sulfate, keratin sulfate, heparin, and acetylheparin sulfate class-one mucoitin substances. The method of introduction may be accomplished by coupling, chemical adsorption, physical adsorption, or collagen membrane inclusion. Coupling is preferred, and coupling agents that may be used include internal diacid anhydrides, oxamide, oxalyl chloride, diepoxides, carbodiimides, and other bifunctional group substances.

Step 7: Washing and cleaning involves rinsing off excessive chemical or bio-agents with purified water.

Step 8: In the sealing and packaging step, the prosthesis is sealed in a dual-layer plastic bag containing physiological saline storage solution.

Step 9: In the sterilization and virus eradication step, the packed product is sterilized under minimum 25 kGy γ-irradiation. This sterilization method has been proven to kill known pathogens, except prions.

Step 4a: An additional “NaOH treatment” step is required between the crosslinking-fixation treatment and the multiform removal of antigens if the cartilage material is from a bovine or ovine source. In this step, the article is immersed in 1N NaOH at 25-50° C. for more than 60 minutes to kill prion viruses that may be present.

Steps 3-7 in the aforementioned treatment processes can be performed in a high permeation reactor. The reactor can be an air-tight vessel furnished with an ultrasonic vibrating device and a vacuum pulse device. Vacuum pulse can be used to remove air inside the cartilage material, and when used in combination with ultrasonic vibration, the reagents can permeate the micropores deep inside the cartilage material to ensure that the material is thoroughly treated with all the necessary reagents, and to ensure that the reaction is consistent inside and out. In this regard, all the treatments in steps 3-7 can be carried out in the same reactor, though different reagents may be used in the different steps.

The advantages of the biological joint cartilage repair piece of the present invention are that it retains the basic structure and components of the cartilage and its connected bone substrate, it possesses multiple activated organic components, and it provides the organic components sufficient stability. In addition to effectively preventing immune rejection, the present invention helps the cartilage adhere to the bone pedestal with (multiple) growth factors and stem cells, inducing the stem cells to divide and proliferate into bone and cartilage cells, functioning to induce cartilage tissue and bone tissue to regenerate in succession. The prosthesis can also be used with added Bone Morphogenetic Protein (BMP) and/or Mesenchymal Stem Cells (MSCs) to accelerate regeneration. The tissue compatibility of the prosthesis of the present invention is good, since after implantation it does not initiate rejection and is able to induce cartilage and bone tissue regeneration in order to achieve regenerative repair of cartilage damage.

### EXAMPLE

Take a fresh and healthy porcine knee joint, place it in 0.1% benzalkonium bromide sterilization fluid, saturate and disinfect for 30 minutes. Then remove, excise foreign matter that is in the area, carefully cut off the ligaments to expose the joint cartilage, and use special tools to cut to a certain size. The cartilage piece is then placed with the bone pedestal B in a high-permeability cell removal reactor, and add an enzyme solution in order to perform enzymolysis for 2-8 hours, selecting a temperature between 18 and 45° C. A combined pepsin-trypsin enzyme solution whose concentration is 40-200 mg of enzyme per liter is used for enzymolysis. After enzymolysis is completed, perform enzyme elution and deactivation, then place the cartilage piece in a high-permeability fixation reactor (which can be the same reactor as above), add fixation solution, react for 8-96 hours at a reaction temperature between 5 and 40° C. The fixation solution contains 0.1-2.0N epoxide, and the epoxide is as indicated in the molecular formula set forth above. The epoxide can be a single epoxide or a double epoxide, and the number of carbon atoms it contains can be selected from 2-12. Once fixation is completed, the cartilage piece is removed, and epoxide neutralized, and then the cartilage piece is washed and cleaned. The cartilage piece is then placed in a high-permeability antigen removal reactor, antigen removal reagent is added, and then the cartilage piece and the reagent is reacted at a particular temperature between 10 and 50° C. for 2-24 hours. The antigen removal reagents used are carboxylic acid anhydrides, acylamides, acyl chlorides, epoxides, and guanidine hydrochloride. Two or more antigen removal reagents are used in succession to perform the reaction in order to fully remove antigenicity. The cartilage piece is then removed and washed, and then placed in a high-permeability tissue induction reactor (which can be the same reactor). Adhesive growth factor and cellular active reagent solution and coupling agent solution are added, and then reacted with the cartilage piece for 2-24 hours at .a reaction temperature between 5-30° C. The active reagent is a polypeptide composed of lysine (16)—glycine-arginine-glycine-aspartic acid-serine-proline-cysteine, the coupling agent is glutaric acid anhydride or a diepoxide. After the reaction is complete, the cartilage piece is removed, washed and cleaned, then sealed and packaged, and then sent to the radiation center for γ-irradiation (25 kGy) sterilization and virus eradication, after which the finished product is obtained.

While the description above refers to particular embodiments of the present invention, it will be understood that many modifications may be made without departing from the spirit thereof. The accompanying claims are intended to cover such modifications as would fall within the true scope and spirit of the present invention.

1-16. (canceled)

17. A cartilage implant made according to a method that comprises the following steps:
collecting animal material from a bovine, ovine or porcine source, the animal material being a cartilage;
shaping the animal material to provide a desired shape for the cartilage implant;
removing cells from the animal material;
crosslinking and fixing the animal material;
blocking residual specific active groups in protein molecules of the animal material after fixation by applying at least one active reagent;
altering the specific conformation of protein molecules of the animal material by a reagent with strong hydrogen bonding power; and
coupling into the animal material active substances which are capable of adhering growth factor and stem cell.

18. The implant of claim 17, wherein the cell removal step uses enzymolysis and/or washing with a surfactant.

19. The implant of claim 17, wherein the crosslinking step is implemented using the epoxy compound 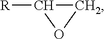
R=CₙH₂ₙ₊₁ group or 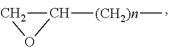
n=0, 1, 2, 3 . . . 12, as the crosslinking agent.

20. The implant of claim 17, wherein the active substances are polypeptides containing 16 lysine oligopeptides with arginine, glycine, and aspartic acid.

21. The implant of claim 17, wherein the at least one active reagent to block specific active groups in the protein molecules of the substrate can be acid anhydrides, acid chlorides, or acylamides.

22. The implant of claim 17, wherein the reagent with strong hydrogen bonding power is a guanidine compound.

23. The implant of claim 17, wherein the animal material is fixed by an epoxy compound that has a hydrocarbon backbone, that is water-soluble, and which does not contain an ether or ester linkage in its backbone.
